(19) United States
(12) Patent Application Publication
(10) Pub. No.: US 2019/0104978 A1
(43) Pub. Date: Apr. 11, 2019

(54) FORCE MEASURING SPECULUM

(71) Applicant: UNIVERSITY OF UTAH RESEARCH FOUNDATION, Salt Lake City, UT (US)

(72) Inventors: Brian K. Cottle, Provo, UT (US); Robert W. Hitchcock, Salt Lake City, UT (US); Stefan Julius Niederauer, Salt Lake City, UT (US)

(21) Appl. No.: 16/154,380

(22) Filed: Oct. 8, 2018

Related U.S. Application Data
(60) Provisional application No. 62/570,010, filed on Oct. 9, 2017.

(51) Int. Cl. A61B 5/22; A61B 5/00; A61B 1/303

(52) U.S. Cl. CPC A61B 5/227; A61B 2562/0261; A61B 1/303; A61B 5/6885

(57) ABSTRACT

The present disclosure relates to a speculum device for measuring pelvic floor muscle (PFM) contractile forces. The speculum device includes a first bill and a second, opposing bill. Each bill has a proximal end attached to a handle. The handle includes a housing in which a sensor cassette may be selectively inserted. When inserted, the sensor cassette can measure contractile forces applied to the upper and lower bills and mechanically transferred to the sensor cassette. The sensor cassette does not come into contact with the patient while measurements are taken. Afterwards, the sensor cassette may be removed from the housing while the remainder of the device is sterilized.

### CROSS-REFERENCE TO RELATED APPLICATIONS

This Application claims priority to and the benefit of U.S. Provisional Patent Application No. 62/570,010, filed on Oct. 9, 2017 and titled “Force Measuring Speculum,” the entirety of which is incorporated herein by this reference.

### BACKGROUND

Pelvic Floor Disorders (PFDs) affect nearly one in four women in the United States (Nygaard, Ingrid et al. “Prevalence of Symptomatic Pelvic Floor Disorders in US Women.” JAMA: the journal of the American Medical Association 300.11 (2008): 1311-1316). These disorders are caused by weakening of the muscles that line the bottom of the abdominal cavity, and can lead to urinary incontinence, fecal incontinence, and pelvic organ prolapse. The pelvic floor muscles form a hammock-like supporting structure for abdominal organs. Inability of the pelvic floor muscles to support the abdominal organs can lead to pelvic floor symptoms, such as urinary incontinence due to lack of support at the bladder neck (DeLancey et. al. (1994) “Structural support of the urethra as it relates to stress urinary incontinence: The hammock hypothesis” American Journal of Obstetrics & Gynecology, Volume 170, Issue 6, 1713-1723).

The voluntary contraction force of the pelvic floor muscles can be measured internally from within the vagina. This measurement is known as the vaginal closure force, and the voluntary contraction is known as a Kegel contraction. The force generated by the pelvic floor muscles during a Kegel contraction, is an important measure of pelvic floor function. Vaginal closure force is the most direct method of evaluating the muscle condition and gives clinicians an objective measurement that can be used to track disease progression and pelvic muscle health.

Physicians conventionally use digital palpation to evaluate the strength of pelvic floor muscles, such as the Brinks test or Oxford scale. Digital palpation is inexpensive and simple, but digital palpation is a subjective measure, and has been shown to have poor reliability for pelvic floor muscle force evaluation (Bo and Finckenhagen (2001), “Vaginal palpation of pelvic floor muscle strength: inter-test reproducibility and comparison between palpation and vaginal squeeze pressure” Acta Obstetricia et Gynecologica Scandinavica, 80: 883-887).

Some devices, such as the balloon perineometer, measure intravaginal closure pressure instead of direct force. Pressure measuring devices are often balloon type devices and have significant compliance when under load. The balloon compliance means that muscle measurements are not isometric, and significant bias may be introduced due to muscle length-tension relationships (Ashton-Miller et al. “Validity and Reliability of an Instrumented Speculum Designed to Minimize the Effect of Intra-Abdominal Pressure on the Measurement of Pelvic Floor Muscle Strength” Clinical biomechanics (Bristol, Avon) 29.10 (2014): 1146-1150).

Other research devices have been manufactured to measure vaginal contraction force (Miller et al. “Test-Retest Reliability of an Instrumented Speculum for Measuring Vaginal Closure Force” Neurourology and urodynamics 26.6 (2007): 858-863; Ashton-Miller, 2014; Chamochumbi et al. (2012) “Comparison of active and passive forces of the pelvic floor muscles in women with and without stress urinary incontinence” Brazilian Journal of Physical Therapy, 16(4), 314-319; Dumoulin et al. (2003) “Development of a dynamometer for measuring the isometric force of the pelvic floor musculature” Neurourol. Urodyn., 22: 648-653; Nunes et al. (2011) “Reliability of bidirectional and variable-opening equipment for the measurement of pelvic floor muscle strength” Phys. Med. Rehab. 3(1):21-6).

While such devices can be used to measure vaginal closure force and detect pelvic floor muscle weakness, each of these devices is designed to directly and permanently fix the vaginal closure force sensing elements to the patient-contacting portion of the device. This can complicate the post-measurement reprocessing of the force measuring device. Because of the potential for patient cross-contamination from reprocessed medical devices, vigilant sterilization procedures must be used. Devices undergoing reprocessing are subjected to extreme environmental conditions, such as heat, humidity, and chemicals. Typical sensing elements used on conventional force measuring devices cannot withstand such conditions and will typically fail after a few reprocessing cycles, severely limiting the usefulness of the devices and making it difficult for clinicians to justify purchasing such devices.

### BRIEF SUMMARY

The present disclosure relates to a force measuring speculum device configured to measure pelvic floor muscle contractile forces. Certain embodiments described herein include a sensor cassette selectively detachable from the patient-contacting portions of the device. These embodiments prevent the sensor componentry included in the sensor cassette from coming into contact with the patient during use of the device. This allows the non-contaminated sensor cassette to be detached and maintained for future use while the contaminated, patient-contacting portion undergoes intensive sterilization to ensure complete sterility before being used with the next patient. Intensive sterilization may include, for example, autoclaving, ethylene oxide treatment, ultraviolet and/or microwave radiation, and combinations thereof. Speculum devices as described herein can be effectively used multiple times, enhancing the longevity of the device and reducing associated per-patient costs.

In one embodiment, a speculum device configured to measure pelvic floor muscle contractile forces includes a bill assembly having a first bill and a second bill. Each of the first bill and second bill include a distal end and a proximal end. The speculum device also includes a handle to which the proximal end of the first bill and the proximal end of the second bill attach. A sensor cassette is selectively attachable/detachable to the handle. The bill assembly is configured to mechanically transfer force to the sensor when the bill assembly is subjected to a contractile force such as resulting from pelvic floor muscle contraction against the bill assembly. The sensor cassette includes a sensor positioned so that when the sensor cassette is coupled to the handle, the contractile force is transferred from the bill assembly to the sensor.

In some embodiment, the handle includes an internal housing. The sensor cassette is configured to fit within the housing through an aperture that allows selective movement of the sensor cassette in and out of the housing. The aperture may be disposed, for example, on an upper side of the handle and/or a proximal end of the handle. In some embodiments, the handle includes a hinge that allows the handle to be pivoted open and away from the bill assembly to reveal the housing. In some embodiments, the handle can be completely detached from the bill assembly.

In some embodiments, the sensor cassette includes an electronics package electrically coupled to the sensor. The electronics assembly may include a wireless communications module for wirelessly communicating force measurements to one or more external computer devices.

In some embodiments, the bill assembly is configured so that the first and second bills remain substantially parallel with one another when the bill assembly is subjected to a contractile force. For example, the first bill may be attached to the second bill via a plurality of linkages that form a parallelogram assembly.

In some embodiments, at least the first bill forms a cantilever, with the proximal end of the first bill being anchored to the handle and the distal end of the first bill being free. The first bill may include a transfer extension that extends proximally from an interior surface of the first bill towards the sensor to enable mechanical transfer of cantilever deflection to the sensor. The sensor may be oriented to measure longitudinally oriented movement of the transfer extension occurring in response to a contractile force applied to the bill assembly. The sensor may alternatively be configured to measure laterally oriented strain of the bill assembly in response to an applied contractile force.

Additional features and advantages will be set forth in part in the description that follows, and in part will be obvious from the description, or may be learned by practice of the embodiments disclosed herein. The objects and advantages of the embodiments disclosed herein will be realized and attained by means of the elements and combinations particularly pointed out in the appended claims. It is to be understood that both the foregoing brief summary and the following detailed description are exemplary and explanatory only and are not restrictive of the embodiments disclosed herein or as claimed.

### BRIEF DESCRIPTION OF THE DRAWINGS

A more particular description of the invention briefly described above will be rendered by reference to specific embodiments which are illustrated in the appended drawings. Understanding that these drawings depict only typical embodiments of the invention and are not limiting of its scope, the invention will be described and explained with additional specificity and detail through the use of the accompanying drawings in which:

FIG. 1 illustrates an isometric view of an exemplary force measuring speculum device including opposing bills and a proximal handle coupled to the bills;

FIG. 2 illustrates a cross-sectional view taken along a longitudinal plane of the speculum device of FIG. 1, showing an internal housing within the handle in which a sensor cassette is selectively inserted;

FIG. 3 illustrates an exploded view of the speculum device of FIGS. 1 and 2;

FIG. 4 illustrates an expanded view of a sensor cassette that may be utilized with the speculum device of FIGS. 1 through 3;

FIG. 5 illustrates an expanded view of an electronics package that may be included with the sensor cassette of FIG. 4;

FIG. 6 illustrates exemplary positioning of the device within the vaginal canal during use of the device to measure PFM contractile force;

FIG. 7 illustrates an alternative embodiment of a force measuring speculum device having a parallelogram assembly;

FIG. 8 illustrates an alternative embodiment of a force measuring speculum device where a sensor cassette is insertable into a corresponding housing on an upper side of a handle and where the sensor is configured to measure longitudinal movement of a transfer extension in response to an applied contractile force;

FIG. 9 illustrates an alternative embodiment of a force measuring speculum device where a sensor cassette is insertable into a corresponding housing on an upper side of a handle and where the sensor is configured to measure lateral strain of the bill assembly in response to an applied contractile force; and

FIGS. 10A and 10B illustrate an alternative embodiment of a force measuring speculum device where an entire handle is selectively attachable/detachable from the bills.

### DETAILED DESCRIPTION

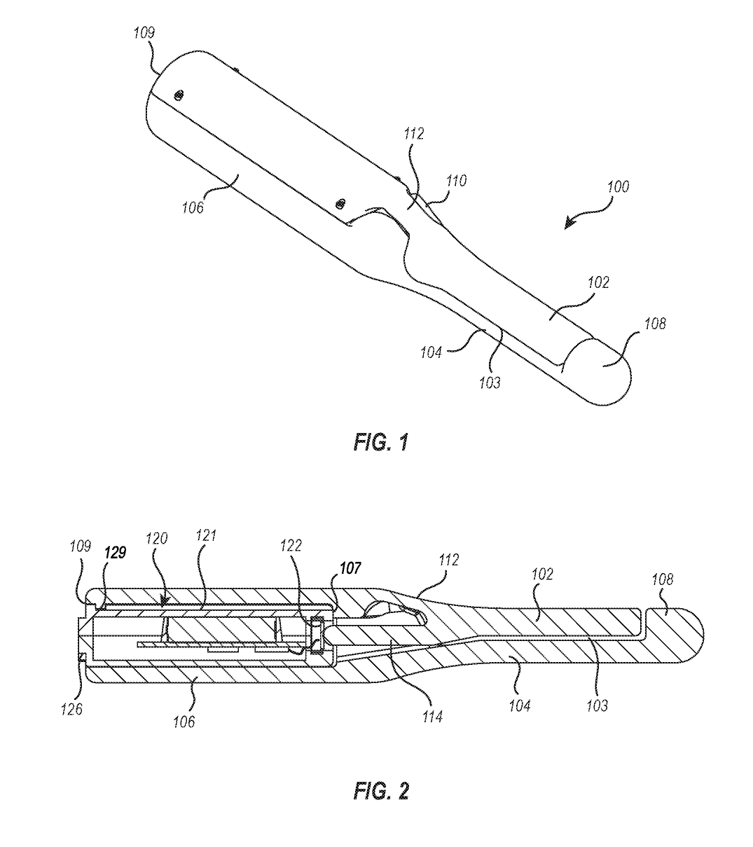

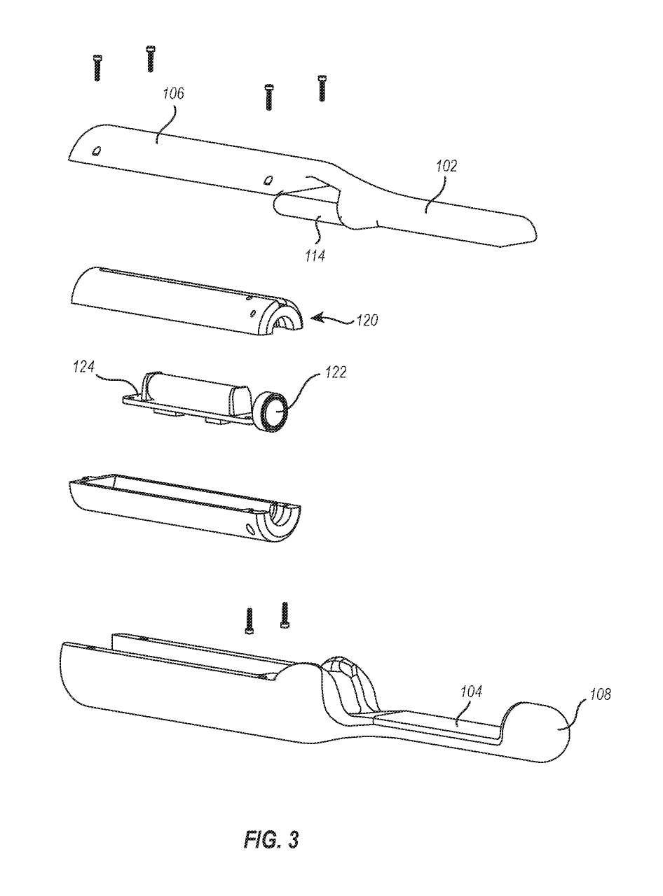

FIG. 1 illustrates an exemplary embodiment of a force measuring speculum device 100 in isometric view. The device 100 is also shown in cross-section in FIG. 2 and in an exploded view in FIG. 3. The device 100 includes a bill assembly including a first bill 102 and an opposing second bill 104. A gap 103 is defined between the first and second bills 102, 104. The bills 102 and 104 each extend between a proximal end and a distal end. The proximal ends of each of the bills 102 and 104 converge to form a handle 106.

In this embodiment, an upper portion of the handle 106 is integrally formed with the first bill 102 and a lower portion of the handle 106 is integrally formed with the second bill 104. This arrangement allows the device 100 to be readily assembled by fastening the upper and lower portions together. In other embodiments, however, one or both of the bills 102, 104 are not necessarily integrally formed with portions of the handle 106, and may be mechanically fastened using other means.

As best shown in FIGS. 2 and 3, the handle 106 includes an internal housing 121 in which a sensor cassette 120 may be placed. The sensor cassette 120 is configured in size and shape to fit within the housing 121. As explained in more detail below, the sensor cassette 120 and housing 121 include features that allow the sensor cassette 120 be locked into place within the housing 121 during active use of the device and to be selectively removed from the housing 121 to allow for sterilization of the patient-contacting portions of the device.

As used herein, “patient-contacting portions” refers to the first and second bills 102, 104, and the outer surfaces of the handle 106. The term does not include the interior portions of the handle where the housing 121 is formed and where the sensor cassette 120 may be positioned. Because the sensor cassette 120 does not contact the patient during use of the device, a clinician or other user can readily reuse the sensor cassette 120 after limited reprocessing (e.g., simple wiping down with a cleaner) as opposed to the intensive sterilization required before reuse of other patient-contacting portions of the device.

The sensor cassette 120 includes a sensor 122 positioned so that when the sensor cassette 120 is placed within the housing 121, the sensor 122 is able to contact a portion of the bill assembly. The bill assembly is configured to mechanically transfer forces to the sensor 122 when the bill assembly is subjected to a contractile force. In the illustrated embodiment, the first bill 102 functions as a cantilever extending from an attached proximal end (e.g., at neck 112) to a free distal end. An extension 114 extends proximally from an inner surface of the first bill 102 toward the sensor 122, and functions to mechanically transfer contractile forces applied to the bill assembly to forces directed to the sensor 122 (e.g., functioning similar to a bell crank).

Other arrangements of the sensor 122 are also possible. For example, the sensor 122 does not necessarily need to be oriented to measure longitudinal movement of the extension 114 and could instead be positioned to directly measure lateral/radial movement of the first bill 102 and/or second bill 104. In some embodiments, the sensor 122 could include a short, longitudinally extending cantilever element that contacts the first bill 102 such that lateral movement of the first bill 102 transfers into corresponding lateral movement in the short sensor cantilever element.

In the illustrated embodiment, the proximal end of the first bill 102 includes a neck 112 that angles radially outward from the rest of the bill 102 and that narrows in width relative to the rest of the bill 102. The proximal end of the second bill 104 may include flanges 110 that broaden in width relative to the rest of the second bill 104. The neck 112 and flanges 110 may cause the first bill 102 to be more readily deflectable than the second bill 104 when a contractile force is applied to the bills 102 and 104. That is, the first bill 102 may tend to deflect by bending at the neck 112 while the second bill 104 remains relatively static. Concentrating the deflective movement primarily to one of the bills can beneficially simplify the transmittal of force to the sensor and can therefore increase measurement fidelity.

The sensor 122 may be any suitable sensor device known in the art capable of translating strain, force, and/or movement in the bill assembly to an electrical signal, including force sensors, pressure sensors, magnetic sensors, optical sensors, and displacement sensors. Examples of such sensors include button-type load cells and other strain gauge load cells, piezoelectric load cells, capacitive load cells, linear variable displacement transducers (LVDTs), force sensing resistors, piezoresistive and capacitive pressure sensors, and optical strain or pressure gauges. A pressure transducer may also be utilized in some embodiments. For example, a pressure transducer filled with fluid may increase in pressure when contacted against a deflecting first bill 102, and the resulting increased pressure readings can be readily correlated to the corresponding forces.

One benefit of the illustrated sensor arrangement is that the sensor 122 may be positioned proximal of the bill assembly. That is, because the internal extension 114 transmits bill deflection into a longitudinal component, the sensor 122 need only be positioned just proximal of the extension 114. In contrast, to directly measure lateral deflection of the bill 102, the sensor 122 would likely have to be positioned distally far enough to directly contact a deflecting portion of the first bill 102. The more the sensor 122 can be located proximally, the less likely it is to be contaminated as a result of coming into contact with the patient or with other portions of the device that come into contact with the patient.

In the illustrated embodiment, the sensor cassette 120 is insertable through an aperture disposed on the proximal end 109 of the handle 106. In this configuration, the housing 121 is a bore extending distally into the handle 106 up to a collar 107 disposed near the proximal end of extension 114. The collar 107 functions to limit distal movement of the sensor cassette 120 past the collar 107. Other embodiments, examples of which are described in more detail below, may include additional or alternative means of inserting the sensor cassette into the housing.

The illustrated embodiment also includes a static distal tip 108. The second bill 104 extends distally past the distal end of the first bill 102 to form the static distal tip 108. As explained in more detail below, the static distal tip 108 functions to support tissue above the pelvic floor so that closure of the upper vagina (above the pelvic floor) due to increases in intraabdominal pressure have limited influence on the deflectable portion of the first bill 102.

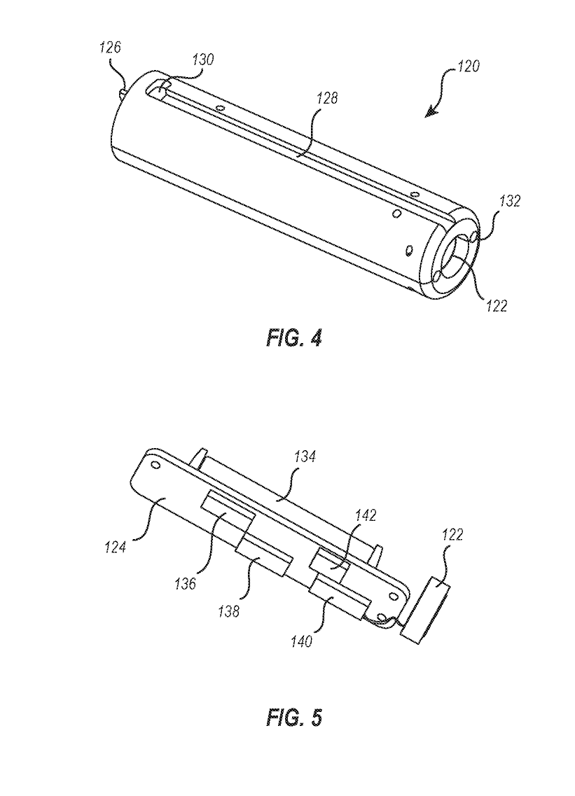

FIG. 4 is an expanded view of the sensor cassette 120. In this embodiment, the sensor cassette 120 includes a longitudinal groove 128 and a connected notch 130 at the proximal end of the groove 128. The groove 128 and notch 130 correspond to a key 129 extending radially inward from an inner surface of the housing 121. This configuration allows the sensor cassette 120 to be readily slid into the housing 121 and then rotated so that the key 129 remains positioned in the notch 130 to lock the cassette 120 in the proper position. A tab 126 may be included on the proximal end of the cassette 120 to provide the user with a greppable structure for rotating and pushing/pulling the cassette 120 into and out of position as desired.

Other embodiments may additionally or alternatively include other mechanisms for coupling the sensor cassette 120 to the handle 106. For example, some embodiments may include additional grooves and corresponding keys on the cassette 120 or in the housing 121. Some embodiments may include a closure cap or clamp that covers the aperture, a cam lever, or features that provide a friction fit, a snap fit, threaded engagement, or a spring-loaded cassette. Some embodiments may include one or more magnets that function to hold the cassette 120 in the housing 121. The illustrated embodiment, for example, includes magnets 132 disposed at the distal end of the cassette 120. Corresponding magnets, and/or ferric materials may be disposed at the collar 107 so as to magnetically hold the cassette 120 in place within the housing 121.

FIG. 5 is an expanded view of the electronics package 124 that may be included in the sensor cassette 120. The electronics package 124 may include a power source 134, such as a battery pack, a signal conditioner 140, a communications module 142, a processor 136, and memory 138. The signal conditioner 140 may be configured to provide signal filtering, amplification, or other signal modulation of the voltage signal received from the sensor 122. The communications module 142 is configured to communicatively link to one or more external computer devices and to coordinate data transfer to/from the computer device(s). In some embodiments, the communications module 142 is configured to coordinate wireless communication, such as via short-wavelength ultra-high frequency radio waves (e.g., Bluetooth®).

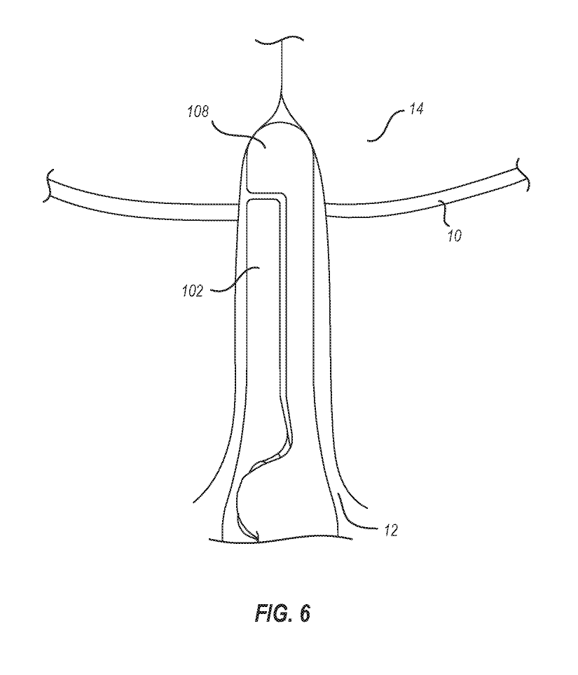

FIG. 6 illustrates exemplary positioning of the speculum device 100 during a pelvic floor muscle contractile force measurement procedure. The device is positioned within vaginal opening 12 until the distal tip 108 is positioned just past the pelvic floor muscles 10 and the first bill 102 is coincident with the pelvic floor muscles 10. The static construction of the distal tip 108 can serve as a buffer that supports the tissue of the upper vagina 14 and limits intraabdominal pressure forces from influencing the first bill 102. In use, proper positioning can be achieved by inserting the device past the pelvic floor 10 and then slowly withdrawing the device while force readings are monitored. A relatively steep drop in measured force will occur just as the distal end of the first bill 102 clears the upper vagina 14 and becomes coincident with the pelvic floor 10.

As shown, the bills of the device are brought into contact with the patient but the more proximally located handle is not. The device is thus preferably dimensioned to minimize the chance of the sensor cassette coming into contact with the patient. The bill assembly preferably has a length of about 4 to 9 inches, or about 5 to 8 inches, or about 6 to 7 inches, with a diameter of about 0.5 to 2 inches, or about 1 inch. The handle 106 may have a length of about 3 to 7 inches, or about 4 to 6 inches. The diameter of the handle is preferably larger than the diameter of the bill assembly in order to provide sufficient room for the housing 121 and to provide more ergonomic size. The diameter of the handle 106 may be about 0.75 inches to 2 inches, for example.

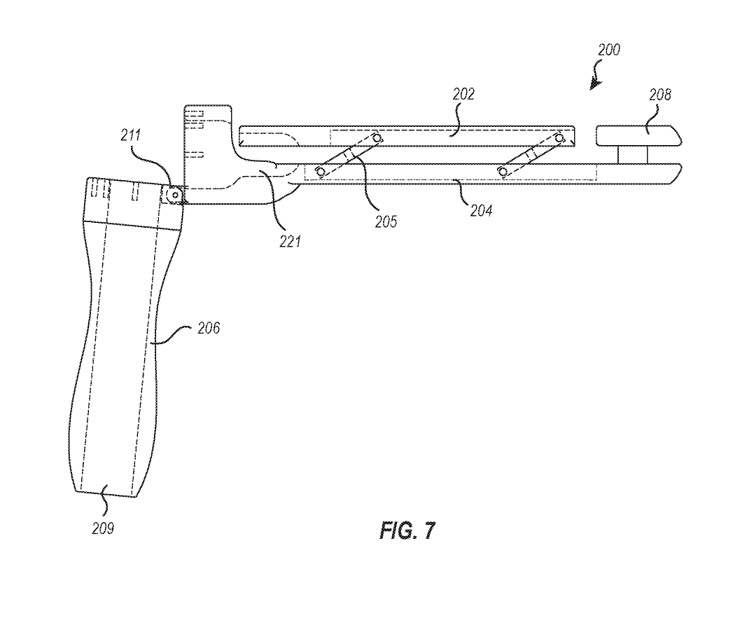

FIG. 7 illustrates an alternative embodiment of a force measuring speculum device 200. The device 200 includes a first bill 202, a second bill 204, and a handle 206. In this embodiment, the first bill 202 and second bill 204 are configured to maintain a parallel orientation. When subjected to a contractile force, the parallelogram linkages 205 ensure the entire length of the first bill 202 moves laterally toward the second bill 204. This arrangement beneficially provides position insensitivity to the device. That is, for a contractile force directed at a given position along the length of the first bill 202, the resulting movement of the first bill 202 relative to the second bill 204 will be substantially the same as if the force were directed at another position along the length of the first bill 202.

As with other embodiments described herein, a sensor or sensor cassette (not shown) may be selectively positioned within housing 221 at a location that avoids or at least minimizes patient contact. The sensor may be passed through proximal aperture 209 into proper position in contact with the bill assembly. The illustrated embodiment also includes a hinge 211 allowing the handle 206 to be pivotably opened to expose the housing 221. The device 200 may also include a static distal tip 208, such as with other embodiments described herein.

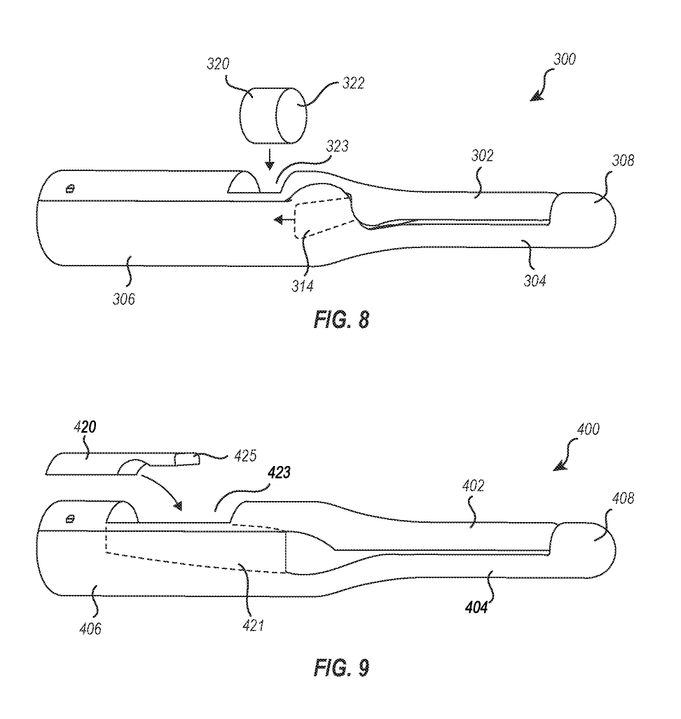

FIG. 8 illustrates another embodiment of a force measuring speculum device 300. The device 300 may share features with other embodiments described herein, and like components include like reference numbers. The device 300 includes a first bill 302, a second bill 304, a handle 306, and a distal tip 308. In this embodiment, the handle 306 includes an aperture 323 disposed on a side surface and the sensor cassette 320 is insertable through the side aperture 323. The sensor component 322 of the cassette 320 may be oriented distally so that extension 314 can transmit longitudinally-oriented forces to the sensor 322 in response to a contractile force applied to the bill assembly.

FIG. 9 illustrates another embodiment of a force measuring speculum device 400. The device 400 may share features with other embodiments described herein, and like components include like reference numbers. The device 400 includes a first bill 402, a second bill 404, a handle 406, and a distal tip 408. As with the embodiment of FIG. 8, this embodiment includes an aperture 423 disposed on a side surface of the handle 406 and the sensor cassette 420 is insertable through the side aperture 423. Unlike the embodiment in FIG. 8, however, the sensing component 425 of the cassette 420 extends so as to be capable of measuring lateral strain/movement of the first bill 402 in response to a contractile force applied to the bill assembly.

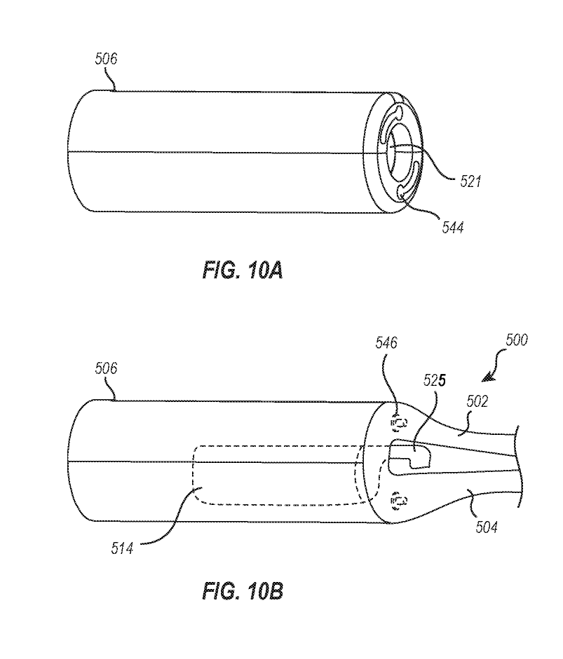

FIGS. 10A and 10B illustrate another embodiment of a force measuring speculum device 500. The device 500 may share features with other embodiments described herein, and like components include like reference numbers. The device 500 includes a first bill 502, a second bill 504, and a handle 506 with an internal housing 521. The handle 506 may be completely and selectively detached from the bill assembly. A sensor cassette 514 with sensor 525 may then be inserted into the housing 521 through the distal end of the handle 506, and the handle 506 may then be reattached to the bill assembly by a suitable fixing means. In the illustrated embodiment, for example, the handle 506 may include one or more notches 544 that match with corresponding pins 546 of the bill assembly. Other selective attachment/detachment mechanisms may additionally or alternatively be used. For example, the handle may utilize magnetic couplings, clasps/clamps, threaded engagement, or other suitable means to attach/detach the handle 506 and bill assembly.

One or more features of a particular embodiment described herein may be combined with any of the other embodiments described herein. For example, features pertaining to a handle configuration of one particular embodiment may be combined with features pertaining to a bill configuration of another embodiment, and vice versa.

The terms “approximately,” “about,” and “substantially” as used herein represent an amount or condition close to the stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a stated amount or condition.

The present invention may be embodied in other forms, without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A speculum device configured to measure pelvic floor muscle contractile forces, the device comprising:
a bill assembly including a first bill having a proximal end and a distal end, and a second bill having a proximal end and a distal end;
a handle to which the proximal end of the first bill and the proximal end of the second bill attach; and
a sensor cassette attachable to the handle and including a sensor,
wherein the bill assembly is configured to mechanically transfer force to the sensor when the bill assembly is subjected to a contractile force, and
wherein the sensor cassette is selectively detachable from the handle.

2. The device of claim 1, wherein the handle includes an internal housing, wherein the sensor cassette is configured to fit within the housing, and wherein the housing includes an aperture to enable selective movement of the sensor cassette in and out of the housing.

3. The device of claim 2, wherein the housing is a bore that extends longitudinally within the handle.

4. The device of claim 2, wherein the aperture is disposed on an upper side of the handle.

5. The device of claim 2, wherein the aperture is disposed on a proximal end of the handle.

6. The device of claim 2, wherein the handle includes a hinge enabling the handle to be pivoted and opened to reveal the housing.

7. The device of claim 2, wherein the handle is selectively detachable from the first and second bills.

8. The device of claim 1, wherein the sensor cassette further comprises an electronics package electrically coupled to the sensor, the electronics assembly including a wireless communications module for wirelessly communicating force measurements to one or more linked computer devices.

9. The device of claim 1, wherein the second bill extends distally beyond the first bill to form a static distal tip, the static distal tip enabling the separation of intraabdominal pressure from pelvic muscle force as applied to the first and second bills.

10. The device of claim 1, wherein the second bill is disposed laterally opposite the first bill.

11. The device of claim 1, wherein the first bill and second bill are configured to remain substantially parallel when the bill assembly is subjected to a contractile force and while the contractile force is mechanically transferred to the sensor.

12. The device of claim 11, wherein the first bill is attached to the second bill via a plurality of linkages to form a parallelogram assembly.

13. The device of claim 1, wherein the first bill forms a cantilever, with the proximal end of the first bill being anchored to the handle and the distal end of the first bill being free.

14. The device of claim 13, wherein the first bill further comprises a transfer extension that extends proximally from an interior surface of the first bill towards the sensor to enable mechanical transfer of cantilever deflection to the sensor.

15. The device of claim 14, wherein the sensor is configured to measure longitudinally oriented movement of the transfer extension occurring in response to a contractile force applied to the bill assembly.

16. The device of claim 13, wherein the proximal end of the first bill includes a neck that angles outward and narrows in width relative to a remaining distal section of the first bill.

17. The device of claim 1, wherein the sensor is configured to measure laterally oriented strain of the bill assembly in response to an applied contractile force.

18. A speculum device configured to measure pelvic floor muscle contractile forces, the device comprising:
a bill assembly including a first bill having a proximal end and a distal end, and a second bill having a proximal end and a distal end, the second bill being disposed laterally opposite the first bill, wherein the second bill extends distally beyond the first bill to form a static distal tip;
a handle to which the proximal end of the first bill and the proximal end of the second bill attach, the handle including an internal housing formed as a bore extending within the handle and including an aperture; and
a sensor cassette configured in size and shape to fit within the housing by passing into the aperture, the sensor cassette including a sensor,
wherein the bill assembly is configured to mechanically transfer force to the sensor when the bill assembly is subjected to a contractile force,
wherein the first bill forms a cantilever, with the proximal end of the first bill being anchored to the handle and the distal end of the first bill being free,
wherein the first bill further comprises a transfer extension that extends proximally from an interior surface of the first bill towards the sensor to enable mechanical transfer of cantilever strain to the sensor, and
wherein the sensor cassette is selectively detachable from the handle.

19. The device of claim 18, wherein the aperture is disposed on a proximal end of the handle.

20. A method of measuring pelvic floor muscle contractile force, the method comprising:
providing a speculum device, the speculum device including
a bill assembly including a first bill having a proximal end and a distal end, and a second bill having a proximal end and a distal end, wherein the second bill extends distally beyond the first bill to form a static distal tip;
a handle to which the proximal end of the first bill and the proximal end of the second bill attach; and
a sensor cassette attachable to the handle and including a sensor,
wherein the bill assembly is configured to mechanically transfer force to the sensor when the bill assembly is subjected to a contractile force, and
wherein the sensor cassette is selectively detachable from the handle;
inserting the speculum device into a vaginal canal so that the static distal tip extends past the pelvic floor and the distal end of the first bill is coincident with the pelvic floor; and
the sensor generating an electrical signal indicative of the contractile force applied to the first and second bills.
